(19) United States
(12) Patent Application Publication
(10) Pub. No.: US 2025/0332333 A1
(43) Pub. Date: Oct. 30, 2025

(54) IMPLANTABLE PUMP DEVICE FOR PUMPING A BODY FLUID

(71) Applicant: B. Braun Miethke GmbH & Co. KG, Potsdam (DE)

(72) Inventors: Gordon Boese, Berlin (DE); Brandon Loudermilk, Potsdam (DE); Christoph Miethke, Potsdam (DE)

(21) Appl. No.: 18/866,532

(22) PCT Filed: May 16, 2023
(86) PCT No.: PCT/EP2023/063169

§ 371 Date: Nov. 16, 2024

(30) Foreign Application Priority Data
May 19, 2022 (EP) 22174262.0

(51) Int. Cl. A61M 1/00; A61M 27/00; A61M 39/24; F04B 19/22

(52) U.S. Cl. CPC A61M 1/81; A61M 27/006; A61M 39/24; F04B 19/22; A61M 2205/0216

(57) ABSTRACT

An implantable pump for pumping body fluid has a piston which is reciprocatingly movable along a stroke axis, with a piston head, a wall opposite the piston head, and a pump volume enclosed between the piston head and the wall. A stroke movement of the piston causes a change in pump volume and delivery of body fluid between an inlet and an outlet. The piston has a disc, the underside of which forms the piston head, and an elastic ring collar that protrudes radially from the disc. An inner periphery of the ring collar is fixedly and fluid-tightly connected to an outer periphery of the disc, and at an outer periphery of the ring collar is fixedly and fluid-tightly connected to the wall. The disc rests via the ring collar on the wall so as to be reciprocatingly movable relative to the wall and fluid-tightly connected to the wall.

### CROSS-REFERENCE TO RELATED APPLICATIONS

This application is the United States national stage entry of International Application No. PCT/EP2023/063169, filed on May 16, 2023, and claims priority to European Application No. 22174262.0, filed on May 19, 2022. The contents of International Application No. PCT/EP2023/063169 and European Application No. 22174262.0 are incorporated by reference herein in their entireties.

### FIELD

The invention concerns an implantable pump device for pumping a body fluid, having a piston which is reciprocatingly movable along a stroke axis, with a piston head, a wall lying opposite the piston head along the stroke axis, and having a pump volume which is enclosed between the piston head and the wall and forms a portion of a fluid path extending between an inlet and an outlet, wherein a stroke movement of the piston causes a change in pump volume and accordingly a pump delivery of body fluid between the inlet and the outlet.

### BACKGROUND

Such an implantable pump device is known for example from U.S. Pat. No. 9,731,101 B2. The known implantable pump device is configured for pumping cerebrospinal fluid (CSF) out of the ventricles of the brain into another body cavity of a patient suitable for receiving the fluid. To this end, the known implantable pump device is implanted for example in the peritoneum and/or the atrium, and connected fluid-conductively by means of suitable catheters to the ventricles on the inlet side and to said cavity on the outlet side. The known implantable pump device works on the displacement principle and may in particular be configured as a peristaltic pump, a screw pump, a diaphragm pump or a piston pump. Piston pumps usually have a movable piston, a wall and a pump volume enclosed between the head of the piston and the wall. The piston is reciprocatingly movable linearly relative to the wall. The stroke movement of the piston is known to lead to a change in pump volume which is associated with a pump delivery between the inlet and outlet. In conventional piston pumps, the wall is formed as a cylinder. During the stroke movement, the piston moves with friction in the inner chamber of the piston, or more precisely along the cylinder inner wall. To seal between the piston and the cylinder inner wall, usually piston sealing means are used which lie on the cylinder inner wall in slidingly movable fashion.

### SUMMARY

The object of the invention is to provide an implantable pump device of the type cited initially which offers advantages relative to the prior art.

This object is achieved in that the piston has a form-stable disc portion, the underside of which forms the piston head, and an elastic ring collar portion, wherein the ring collar portion protrudes radially from the disc portion, at its inner periphery is fixedly and fluid-tightly connected to an outer periphery of the disc portion, and at its outer periphery is fixedly and fluid-tightly connected to the wall, whereby the form-stable disc portion rests via the elastic ring collar portion on the wall so as to be reciprocatingly movable relative thereto and is fluid-tightly connected thereto. Because of the solution according to the invention, a simple structure and particularly robust function are achieved. Associated therewith, the service life of the implantable pump device can be extended and its failure risk reduced. This leads to improved patient safety. In addition, the simple structure allows low-cost production and simplified installation. The above-mentioned advantages are achieved by the design of the piston according to the invention and its cooperation with the wall. The piston structure according to the invention firstly provides the form-stable disc portion and secondly the elastic ring collar portion. The disc portion functions primarily as a displacement element of the implantable pump device working on the displacement principle. In particular, to this end the disc portion is form-stable. The form stability allows the transfer of forces necessary for pumping the body fluid. The ring collar portion serves primarily for fluid-tight sealing and reciprocatingly movable support of the disc portion on the wall. The ring collar portion thus has a particularly advantageous multiple function. This contributes decisively to a simplified structure and robust function of the implantable pump device. To support the disc portion on the wall, the ring collar portion is connected firstly fixedly to the disc portion and secondly fixedly to the wall. In order to allow the necessary stroke movement of the piston despite the fixed connection, the ring collar portion is elastic. Accordingly, during the stroke movement of the piston, the ring collar portion is elastically deformed. Because of the fixed connection of the ring collar portion at its inner periphery on one side and its outer periphery on the other, at said points no movement relative to the disc portion or wall takes place. This avoids friction losses. To seal the variable pump volume enclosed between the underside of the disc portion and the wall, the above-described fixed connection between the inner periphery of the ring collar portion and the outer periphery of the disc portion on one side, and the outer periphery of the ring collar portion and the wall on the other, is also fluid-tight. Thus in particular, no separate sealing elements are required. The fixed and fluid-tight connections may be configured as substance-bonded, form-fit and/or force-fit connections, or by means of integral cohesive production. The form-stable properties of the disc portion and the elastic and/or form-flexible properties of the ring collar portion are achieved by a corresponding dimensioning of the thicknesses and choice of material. Preferably, the disc portion is thick in comparison with the ring collar portion. Preferably, the ring collar portion is thin in comparison with the disc portion. Preferably, the difference between the thicknesses amounts to at least one order of magnitude, particularly preferably at least two orders of magnitude. Further preferably, a radial extent between the inner periphery and outer periphery of the ring collar portion is at least 3 times, preferably at least 5 times, particularly preferably at least 10 times as large as the thickness of the ring collar portion. In a preferred embodiment, the radial extent of the ring collar portion amounts to 2 mm to 6 mm, particularly preferably 3 mm. In a preferred embodiment, the disc portion has a diameter of 14 mm to 20 mm, particularly preferably 16 mm. The fluid path of the implantable pump device has the inlet at one end and the outlet at the other end. The pump volume forms a portion of the fluid path and is arranged between the inlet and the outlet in the delivery direction of the body fluid. The inlet and the outlet are connected together fluidically via the pump volume. In one embodiment, control elements for controlling the pump delivery, preferably in the form of check valves, are arranged in the fluid path. In a further embodiment, the implantable pump device has no such control elements. Instead, corresponding control elements are arranged for example before the inlet and/or after the outlet, away from the implantable pump device, preferably in a fluid conduction system or similar which can be connected to the implantable pump device. In one embodiment, for driving the stroke movement, the implantable pump device has an actuator which is actively connected to the piston. The actuator is actively connected to the piston, in particular the disc element, preferably force-and/or movement-transmissively. The active connection may be mechanical and/or fluidic. In principle, also a magnetic active connection is conceivable. In a further embodiment, the implantable pump device has no such actuator, wherein instead the actuator can be connected to the implantable pump device for example as a constituent of a separate drive device.

Advantageously, the implantable pump device according to the invention is particularly suitable for pumping body fluids, in particular cerebrospinal fluid. Evidently, the solution according to the invention is equally suitable for pumping medicinal fluids.

In one embodiment of the invention, the disc portion and the ring collar portion are formed integrally and cohesively with one another. Because of the integral design, the inner periphery of the ring collar portion is fixedly and fluid-tightly connected to the outer periphery of the disc portion, and vice versa. Accordingly, no separate joint connection is required. This leads to a further simplified structure and further improved robustness. In this embodiment of the invention, the piston may for example be produced as a turned part, a casting or formed part. Alternatively, additive manufacturing is conceivable.

In a further embodiment of the invention, the disc portion and the ring collar portion are each made of metal, preferably titanium. The inventors have found that this may achieve particular advantages. Firstly, production from a metal, preferably titanium, leads to biocompatible properties. Secondly, there are production advantages. This applies to both a multipiece and also an integrally cohesive production of the disc and ring collar portions. Preferably, the wall is also made of metal, preferably also titanium. In combination with production of the ring collar portion of metal, particular production advantages are achieved. For example, in this way the connection between the outer periphery of the ring collar portion and the wall can be made reliably firm and fluid-tight by simple means.

In a further embodiment of the invention, the outer periphery of the ring collar portion is joined fixedly and fluid-tightly to the wall by means of a substance-bonded joint connection. In one embodiment, the substance-bonded joint connection is an adhesive connection. In a further embodiment, a weld connection is provided. This design of the invention leads to further production advantages. In addition, the structure of the implantable pump device is further simplified since no separate connecting means are required for connecting the outer periphery of the ring collar portion to the wall.

In a further embodiment of the invention, the substance-bonded joint connection is a weld connection, preferably produced without a welding additive. In this case, the ring collar portion and the wall are preferably each made of metal or weldable plastic. In comparison with an adhesive connection for example, the weld connection offers better biocompatible properties. This applies in particular if the weld connection is produced without welding additive. In this case, the weld connection is for example a laser or friction weld, or a resistance weld connection.

In a further embodiment of the invention, a thickness and/or a shaping of the ring collar portion is configured such that a stroke-induced elastic deformation of the ring collar portion causes a spring force opposite the stroke movement, wherein the spring force causes or at least supports a complete return of the piston. Accordingly, in this embodiment, the ring collar portion also functions as a type of spring element for returning the piston. Thus, no separate structural elements are required for returning the piston. At least such components may be dimensioned less strongly. In other words, the ring collar portion in this embodiment preferably functions as a type of cup spring.

In a further embodiment of the invention, the ring collar portion has a flat shaping. Accordingly, the ring collar portion is flat in the radial direction and/or in the circumferential direction. Such a flat form supports simple production. Also, production of the fixed and fluid-tight connection between the outer periphery of the ring collar portion and the wall is facilitated by the flat shaping.

In a further embodiment of the invention, the ring collar portion has an undulating shaping. The wave form runs in the radial direction and/or in the circumferential direction of the ring collar portion. Preferably, the ring collar portion has multiple radial undulations. In other words, over its radial extent, the ring collar portion has multiple undulations which extend continuously in the circumferential direction. The inventors have found that the undulating shaping in particular supports a suitable elastic deformation of the ring collar portion. This supports an easy but nonetheless adequately supported and/or guided upward and downward movement of the disc portion connected to the ring collar portion.

In a further embodiment of the invention, the wall has a further elastic ring collar portion, the outer periphery of which is fixedly and fluid-tightly connected to the outer periphery of the elastic ring collar portion of the piston. The further ring collar portion improves the deformation behavior of the ring collar portion of the piston. The improved deformation behavior is associated with a particularly easy reciprocating moveability of the disc portion. This supports a particularly energy-efficient operation of the implantable pump device. Preferably, the further ring collar portion protrudes radially outward from a middle portion of the wall opposite the disc portion. In this embodiment of the invention, the pump volume is preferably enclosed between the underside of the disc portion and said middle portion. The ring collar portion of the disc element and the further ring collar portion of the wall lie on one another at their outer peripheries and are there fixedly and fluid-tightly connected together. Here again, a connection by substance bonding, form fit and/or force fit is used. A weld connection is preferred.

In a further embodiment of the invention, the ring collar portion of the piston and the further ring collar portion of the wall are arranged and/or configured mirror-symmetrically relative to a radial center longitudinal plane of the pump volume. Said mirror symmetry may firstly offer production advantages. Secondly, it leads to a particularly advantageous elastic deformation behavior of the two ring collar portions and hence a particularly suitable reciprocating movability of the disc portion.

In a further embodiment of the invention, the fluid path has a first check valve which is arranged between the inlet and the pump volume, and a second check valve which is arranged between the pump volume and the outlet and opens and closes in the opposite direction to the first check valve. The first check valve can also be described as an inlet valve. The second check valve can also be described as an outlet valve. Because of their design, the two check valves allow a particularly simple structure of the implantable pump device. This also allows a particularly robust control—hence not susceptible to faults—of the pump delivery of body fluid. In a further embodiment, instead of check valves, electronic control valves or similar are provided.

In a further embodiment of the invention, the first check valve and/or the second check valve are integrated in the wall. This gives various structural advantages. Also, in comparison with integration in the piston—which is conceivable in principle—a reduction in oscillating masses is achieved. Secondly, existing installation space can be better utilized. This allows a particularly compact structure of the implantable pump device.

The object according to the invention is also achieved in that a portion of the fluid path extending between the inlet and the pump volume adjoins a back of the piston which lies opposite the piston head along the stroke axis, whereby the piston back is pressurized with an inlet-side pressure of the body fluid. Thanks to this solution according to the invention, pressure fluctuations can be compensated with surprisingly simple means. In the solutions known from the prior art, the piston back is usually encapsulated against a prevailing ambient pressure. Accordingly, fluctuations in ambient pressure act via the body fluid of the patient and from there only on the piston head. With implantable devices known from the prior art, this may lead to an increased force requirement for the stroke movement of the piston. This is associated with an increased energy consumption, which is disadvantageous for various reasons. With this solution according to the invention, the inlet-side pressure of the body fluid—and hence indirectly the ambient pressure—acts on the piston back. Accordingly, to pump the body fluid, only the differential pressure prevailing between the pump volume and the outlet need be overcome. This leads to a relatively easy stroke movement and allows a particularly energy-efficient operation of the implantable pump device. Insofar as the implantable pump device is configured according to one of the preceding embodiments, the piston back is a top side of the form-stable disc portion lying opposite the underside along the stroke axis. The portion of the fluid path adjoining the piston back and/or the top side of the disc portion may be described as the compensation chamber. During operation of the implantable pump device, the body fluid flows from the inlet through the compensation chamber, from there into the pump volume, and from there is conveyed further in the direction of the outlet. In other words, during operation of the implantable pump device, body fluid constantly flows through the compensation chamber. Thus, standing fluid with its associated disadvantages is avoided. In particular, agglomerations on the piston back and resulting germ formation or other health-damaging phenomena are countered.

In a further embodiment of the invention, the implantable pump device has a housing with a first inner chamber containing the piston, the wall and the fluid path, and with a second inner chamber which is sealed fluid-tightly against an environment and the first inner chamber and is configured to receive further components of the pump device. The further components may for example be an actuator for driving the stroke movement of the piston, and/or a control device for controlling the actuator.

The invention furthermore concerns an implantable device with a pump device according to any of the preceding embodiments. The implantable device in one embodiment is a dialysis device for use in cerebral microdialysis, wherein the pump device is configured for pumping cerebrospinal fluid. In a further embodiment, the implantable device serves for pain therapy and/or the treatment of spasticities.

### BRIEF DESCRIPTION OF THE DRAWINGS

Further features and advantages of the invention arise from the following description of preferred exemplary embodiments of the invention which are illustrated in the drawings.

FIG. 1 shows a schematic, greatly simplified, sectional illustration of an embodiment of an implantable device according to the invention, which is provided with an embodiment of an implantable pump device according to the invention;

FIG. 2 shows the implantable pump device from FIG. 1 in a perspective, sectional, exploded view;

FIG. 3 shows a schematic, greatly simplified, sectional illustration of a piston, a wall and a pump volume enclosed between these components, of the implantable pump device from FIGS. 1 and 2;

FIG. 4 shows a variant of the arrangement in FIG. 3

FIG. 5 shows a further variant of the arrangement in FIG. 3.

### DETAILED DESCRIPTION

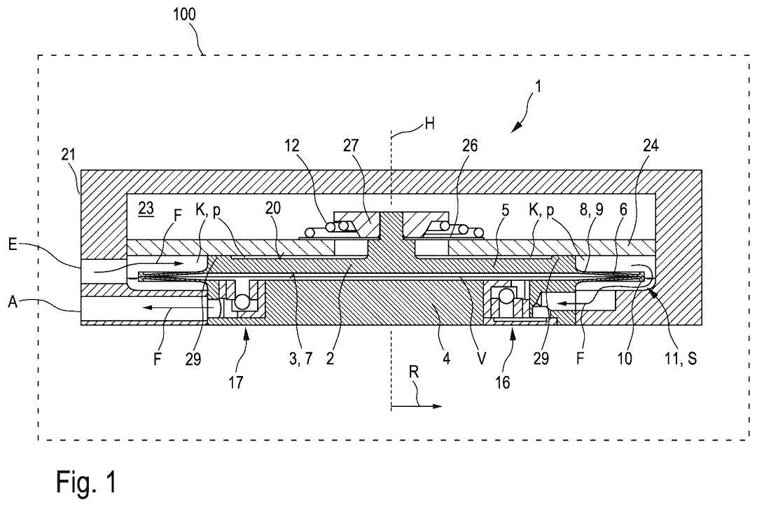

According to FIG. 1, a device 1 is provided which can be implanted in the body of a patient for pumping a body fluid.

In the embodiment shown in FIG. 1, the implantable pump device 1 is part of a device 100, illustrated schematically in simplified form. The implantable device 100 in this case is provided for use in cerebral microdialysis. To this extent, the implantable pump device 1 here serves for pumping cerebrospinal fluid. Alternatively, the implantable device 100 may be used for example in pain therapy and/or for treatment of spasticities.

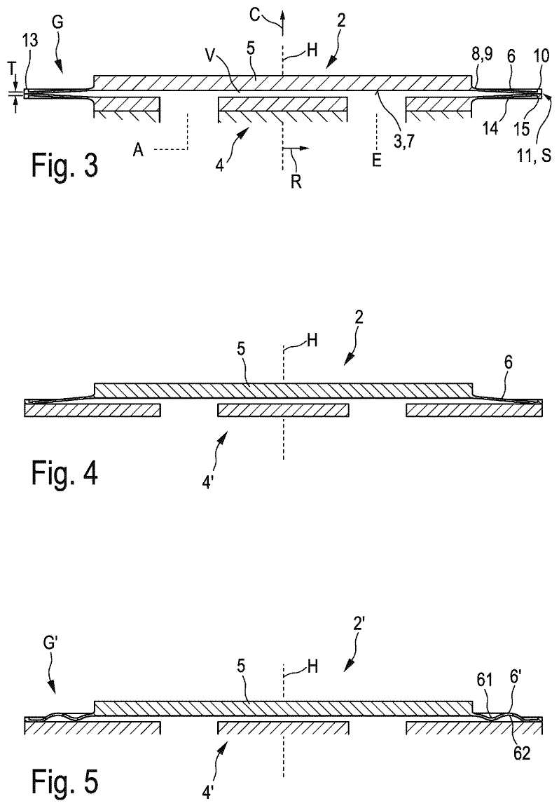

The implantable pump device 1 (referred to below in brief as the pump device) has a piston 2 moving reciprocatingly along a stroke axis H, with a piston head 3, a wall 4 lying opposite the piston head 3 along the stroke axis H, and a pump volume V enclosed between the piston head 3 and the wall 4 (see also FIG. 3). The pump volume V forms a portion of a fluid path F extending between an inlet E and an outlet A of the pump device 1. A linear stroke movement of the piston 2 along the stroke axis H causes a change in pump volume V and accordingly a pump delivery of the body fluid between the inlet E and the outlet A. On a downward movement of the piston 2 (relative to the drawing plane of FIG. 1), the pump volume V is reduced. On an upward movement of the piston 2, the pump volume V is enlarged. Said upward movement causes body fluid to be drawn into the pump volume V. Said downward movement causes the body fluid to be expelled from the pump volume V.

To control the above-described pump delivery along the flow path F, the pump device 1 in the embodiment shown has control elements, to be described in more detail below.

The piston 2 has a form-stable disc portion 5 and an elastic ring collar portion 6. An underside 7 of the disc portion 5, facing the wall 4 along the stroke axis H, forms the piston head 3. The elastic ring collar portion 6 protrudes outwardly from the disc portion 5 in the radial direction R. The ring collar portion 6 runs continuously around in the circumferential direction of the disc portion 5. The ring collar portion 6 has an inner periphery 8 and an outer periphery 10. The inner periphery 8 may also be described as the ring inner edge or inner ring edge. The outer periphery 10 may also be described as the ring outer edge or outer ring edge. The inner periphery 8 is fixedly and fluid-tightly connected to an outer periphery 9 of the disc portion 5. The outer periphery 9 of the disc portion 5 may also be described as the disc outer edge or outer disc edge. The outer periphery 10 of the ring collar portion 6 is fixedly and fluid-tightly connected to the wall 4. Thus, the form-stable disc portion 5 is supported (indirectly) on the wall 4 by means of the elastic ring collar portion 6, firstly so as to be reciprocatingly movable linearly along the stroke axis H relative to the wall 4. Secondly, the form-stable disc portion 5 is (indirectly) connected fluid-tightly to the wall 4 by means of the ring collar portion 6.

In the present case, a radial extent between the inner periphery 8 and the outer periphery 10 of the ring collar portion 6 is significantly greater-namely by at least one order of magnitude-than an axial extent and/or thickness of the ring collar portion 6. In exemplary embodiments not shown in the Figures, the radial extent is at least 3 times greater than the thickness.

On pump delivery of the body fluid, the form-stable disc portion 5 moves linearly up and down along the stroke axis H. Because of its form-stable design, the disc portion 5 undergoes no, or in any case no significant, elastic deformation. This guarantees in particular that pump forces necessary for pump delivery of the body fluid are suitably conducted into the disc portion 5 and can be transmitted from this to the pump volume V. In contrast to the form-stable disc portion 5, the ring collar portion 6 undergoes an elastic deformation during the stroke movement. On a downward movement of the piston 2 and hence of the disc portion 5, the ring collar portion 6 is elastically deformed at its inner periphery 8 along the stroke axis H in the direction of the wall 4. The outer periphery 10 here remains immovable relative to the wall 4. The elastic deformability of the ring collar portion 6 guarantees that the disc portion 5 can move linearly up and down along the stroke axis H. The fluid-tight connection both at the inner periphery 8 and also at the outer periphery 10 guarantees a reliable fluid-tight seal between the piston 2 on one side and the wall 4 on the other. In other words, the ring collar portion 6 functions firstly as a type of bearing and/or support element and secondly as a type of sealing element.

In order to achieve a suitable elastic deformability of the ring collar portion 6, in the embodiment shown, this has a small thickness (to be described in more detail below) in comparison with the disc portion 5. In other words, the disc portion 5 is thick in comparison with the ring collar portion 6. Conversely, in comparison with the disc portion 5, the ring collar portion 6 is thin.

In the embodiment shown, the disc portion 5 has a flat circular cylindrical shape relative to the stroke axis H. The outer periphery 9 of the disc portion 5 is thus circular or round. In the embodiment shown, the ring collar portion 6 protruding radially outward from the disc portion 5 has the form of a circular ring. Such a circular shape of the disc portion 5 and the ring collar portion 6 is not, however, absolutely necessary. In an embodiment not illustrated, the disc portion 5 and ring collar portion 6 are instead each oval.

In the embodiment shown, the disc portion 5 and ring collar portion 6 are formed integrally cohesive with one another. Accordingly, the disc portion 5 and the ring collar portion 6 are different portions of one and the same component. Because of the integrally mutually cohesive design, there is no need for a separate substance-bonded, force-fit and/or form-fit joint connection between the inner periphery 8 of the ring collar portion 6 and the outer periphery 9 of the disc portion 5. This simplifies construction and manufacture. Also, the necessary mechanical and fluid-tight connection between the inner periphery 8 and outer periphery 9 can be formed particularly robust and reliable.

Said integral design is particularly advantageous, but not absolutely necessary for implementation of the present invention. Accordingly, in an embodiment not shown in the Figures, a multipiece production of the piston is proposed, wherein the disc portion and ring collar portion are produced as separate components and then fixedly and fluid-tightly joined together at the inner and outer peripheries by means of a suitable joint connection.

In the embodiment shown, the disc portion and the ring collar portion are made of metal. In particular, production from titanium is provided. Production from metal, in particular titanium, is associated with suitable biocompatibility.

In an embodiment not shown in the Figures, instead of metal, a biocompatible plastic is used to produce the disc portion, ring collar portion and/or piston. Biocompatible plastics are known to the person skilled in the art.

In the embodiment shown, the outer periphery 10 of the ring collar portion 6 is fixedly and fluid-tightly joined to the wall 4 by means of a substance-bonded joint connection 11. The substance-bonded joint connection 11 is configured differently in different embodiments. In the embodiment shown, the substance-bonded joint connection 11 is a weld connection S.

Depending on prevailing material choice, the weld connection S for the piston 2 and wall 4 may be a metal or a plastic weld connection.

In the embodiment shown, the wall 4 is made of metal, in particular titanium. The weld connection S is accordingly a metal weld connection.

In the embodiment shown, the weld connection S is produced without the use of a welding additive. This achieves further advantages. Suitable welding methods for forming the weld connection S are for example laser welding, friction welding and/or resistance welding.

With further reference to FIG. 3, a thickness T and/or a shaping G of the ring collar portion 6 is configured such that a stroke-induced elastic deformation of the ring collar portion 6 causes a spring force C opposite the stroke movement, wherein the spring force C causes or at least supports a complete return of the piston. On a downward movement of the piston 2, the ring collar portion 6 is elastically pretensioned. The elastic pretension of the ring collar portion 6 causes said spring force C. This acts opposite the downward movement of the piston 2. In other words, the elastically deformed ring collar portion 6 pushes the form-stable disc portion 5 back along the stroke axis H into its starting position. Depending on the dimensioning of the thickness T and the specific shaping G, the return is either caused exclusively or is at least supported by the elastic deformation of spring force C. In this case, the latter is provided, wherein an (additional) spring element 12 is present for returning the piston and is actively connected to the piston 2 in a fashion to be described (see FIG. 1).

In the embodiment shown, the thickness T amounts to 0.07 mm. In embodiments not shown in the Figures, the thickness is between 0.04 mm and 0.15 mm.

The ring collar portion 6, starting from its inner periphery 8, extends radially in the direction of its outer periphery 10. This radial extent in this case amounts to 3 mm. In embodiments not shown in the Figures, the radial extent is between 2 mm and 6 mm.

In the present case, the radially extending ring collar portion 6 is at least substantially flat.

In the embodiment shown, the ring collar portion 6 has an axial slope, i.e. the inner periphery 8 and outer periphery 10 have a mutual axial spacing. This axial spacing in the embodiment shown amounts to 0.3 mm. In embodiments not shown in the Figures, the axial spacing is between 0.1 mm and 0.5 mm.

In the embodiment shown, a thickness (not described in detail) of the form-stable disc portion amounts to 1.5 mm. In embodiments not shown in Figures, the thickness of the disc portion is between 1.0 mm and 5.0 mm. Furthermore, in the present case, the disc portion 5 has a diameter of 16 mm. In embodiments not shown in the Figures, the diameter is between 14 mm and 20 mm.

In the present case, in the region of its outer periphery 10, the elastic ring collar portion 6 has an erect axial shoulder 13 along the stroke axis H. The axial shoulder 13 simplifies the formation of the weld connection S. Also, the axial shoulder 13 supports the elastic deformation behavior of the ring collar portion 6.

Still with reference to FIG. 3, the shaping G of the ring collar portion 6 in the present case is configured flat in the radial direction R and also in the circumferential direction. This means there are no protrusions, depressions, undulations or similar on the ring collar portion 6.

In the embodiment shown in FIGS. 1 to 3, the wall 4 also has an elastic ring collar portion 14. This is referred to below as the further ring collar portion 14. The further ring collar portion 14 protrudes outward in the radial direction R and has an outer periphery 15. The outer periphery 15 of the further ring collar portion 14 is fixedly and fluid-tightly connected to the outer periphery 10 of the elastic ring collar portion 6 of the piston 2. Accordingly, said joint connection 11, or more precisely the weld connection S, is formed between the outer periphery 10 of the ring collar portion 6 of the piston 2 and the outer periphery 15 of the further ring collar portion 14 of the wall 4. Because of the further elastic ring collar portion 14, the wall 4 is elastically flexible in portions, namely in the region of the further ring collar portion 14. Thus, during its stroke movement along the stroke axis H, the disc portion 5 is supported by springs effectively connected in series. The inventors have found that the presence of the further ring collar portion 14 brings various advantages. In particular, a suitable reciprocating movability of the piston 2 is supported.

In the embodiment shown, the ring collar portion 6 of the piston 2 and the further ring collar portion 14 of the wall 4 are arranged and configured mirror-symmetrically relative to a radial center longitudinal plane of the pump volume V. The wall 4 with its further ring collar portion 14 may also be described as a further piston. This is because the wall with its further ring collar portion 14 in this case is formed largely identically to the piston 2, wherein a main difference lies in the arrangement of the pistons, firstly movable relative to the stroke axis H and secondly stationary relative thereto except for the further ring collar portion 14. The wall 4 may therefore also be described as a fixed wall.

FIGS. 4 and 5 show variants of the arrangement in FIG. 3. Only essential differences of the variants are described below. Otherwise, reference is made to the disclosure above.

It is clear from the variant in FIG. 4 that the further ring collar portion 14 in FIG. 3 is not absolutely necessary. Accordingly, the wall 4′ there has no such ring collar portion. Apart from this, the piston 2 of the arrangement in FIG. 4 is identical to the piston 2 in FIG. 3.

The variant in FIG. 5 differs from the variant in FIG. 4 by an undulating shaping G′ of the ring collar portion 6′. The undulating shaping G′ of the ring collar portion 6′ is associated with axially extended depressions 61 and protrusions 62. The depressions 61 and protrusions 62 are continuously elongate in the circumferential direction of the ring collar portion 6. Alternatively, the ring collar portion 6′ could be described as having undulations, more precisely radial undulations. The inventors have found that the undulating shaping G′ allows a better reciprocating movability of the disc portion 5 in comparison with the variant in FIG. 4.

In the variant shown in FIG. 5, the axial extent of the depressions 61 and protrusions 62 amounts to 0.4 mm. Said axial extent may also be described as the undulation height of the undulating shaping G′. In variants not shown in the Figures, the axial extent (undulation height) is between 0.2 mm and 0.8 mm. In the variant shown, the inner periphery and outer periphery of the ring collar portion 6′ are spaced apart from one another axially by said undulation height.

In the embodiment shown, check valves 16, 17 are provided as the above-mentioned control elements for controlling the pump delivery. The check valves 16, 17 are arranged in the fluid path F. The check valve 16 may be described as the first check valve or inlet valve. The check valve 17 may also be described as the second check valve or outlet valve. The first check valve 16 is arranged in the fluid path F between the inlet E and the pump volume V. The second check valve 17 is arranged in the fluid path F between the pump volume V and the outlet A. The closing/opening directions of the two check valves 16, 17 are opposite to one another. The first check valve 16 opens on an upward movement of the piston 2 and closes on a downward movement thereof. The second check valve 17 opens on a downward movement of the piston 2 and closes on an upward movement thereof.

In the embodiment shown, the two check valves 16, 17 are integrated in the wall 4. To this end, the wall 4 has a first receiving recess 18 and a second receiving recess 19. The receiving recesses 18, 19 are sunk into the wall 4 along the stroke axis H and each configured to receive one of the check valves 16, 17. The first receiving recess 18 receives the first check valve 18. The second receiving recess 19 receives the second check valve 17.

In an embodiment not shown in the Figures, at least one of the two check valves is integrated in the piston 2. In a further embodiment not shown, both check valves are integrated in the piston 2.

Still with reference to FIG. 1, in the embodiment shown, the fluid path F runs at least in portions along a piston back 20 of the piston 2. In other words, the fluid path F has a portion K (see FIG. 1) which directly adjoins the piston back 20 of the piston 2. The portion K is arranged between the inlet E and the pump volume V. In this case, the portion K lies upstream of the first check valve 16 in the flow direction. On pump delivery of the body fluid, this flows from the inlet E along the fluid path F into the portion K and from there via the first check valve 16 into the pump volume V. The piston back 20 is accordingly pressurized with an inlet-side pressure p of the body fluid to be conveyed. In the embodiment shown, this pressurization acts substantially on an entire surface of the piston back 20, which in this case also comprises the outsides of the ring collar portion 6 facing the portion K. Because of the pressure p prevailing in the portion K, the piston 2 is force-loaded along the stroke axis H in the direction of the wall 4. This force-loading causes a pressure compensation and allows a particularly energy-efficient operation of the pump device 1, as will be explained in more detail below.

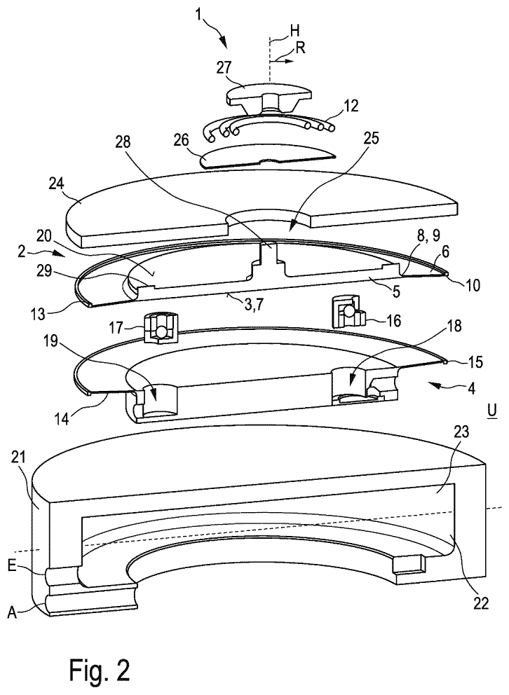

In the embodiment shown, the pump device has a housing 21. The housing 21 is subdivided into a lower half and an upper half. This subdivision is illustrated schematically by the dotted line shown in FIG. 2. Said subdivision divides a complete interior (not designated in detail) of the housing 21 into a first inner chamber 22 and a second inner chamber 23. The first inner chamber 22 contains the piston 2, the wall 4 and the fluid path F. The second inner chamber 23 is sealed fluid-tightly and/or pressure-tightly against an environment U and the first inner chamber 22. The second inner chamber 23 serves to receive further components of the pump device 1. For example, an actuator for driving the pump movement of the piston 2 may be arranged in the second inner chamber 23. Alternatively or additionally, a control electronics for controlling the actuator and/or the stroke movement may be arranged in the second inner chamber 23. Details in this respect are not absolutely necessary for implementation of the present invention, so no further statements are given.

In the embodiment shown, a partition wall 24 is provided for fluid-tight and/or pressure-tight sealing of the second inner chamber 23 from the environment U and the first inner chamber 22. The partition wall 24 subdivides the complete interior of the housing 21 into said lower and upper halves or first and second inner chambers 22, 23. The partition wall 24 in this case has a circular disc shape with a central passage 25. The passage 25 serves for axial passage of a peg 28 which, starting from the piston back 20, protrudes axially upward from the piston 2, i.e. in the direction of the second inner chamber 23. In the embodiment shown, the peg 28 is connected integrally to the other portions of the piston 2. The peg 28 is configured for active connection to an actuator for transmission of force and movement. In other words, the peg 28 serves to introduce pump forces into the piston 2. Said pump forces are generated for example by the above-mentioned actuator arranged inside the second inner chamber 23. The peg 28 penetrates through the passage 25 in the axial direction and is sealed by means of a sealing disc 26. In the embodiment shown, the sealing disc 26 is made of metal, more precisely titanium. In this case, the sealing disc 26 has membrane-elastic properties and may therefore also be described as a sealing membrane.

In the mounted state shown in FIG. 1, the peg 28 fits axially into a receiving bore (not specifically designated) of a pressure piece 27. The pressure piece 27 serves to apply said pump forces and its underside rests on a top side (not specifically designated) of the partition wall 24, with the spring element 12 against the downward movement of the piston 2. The spring element 12 in this case rests indirectly on the partition wall, namely via the sealing disc 26.

It is clear from FIG. 1 that the entire fluid path F extends away and separately from the second inner chamber 23. Because of the fluid-tight and/or pressure-tight encapsulation of the second inner chamber 23, it is not absolutely necessary for the components arranged therein to be biocompatible. Conversely, the components and/or portions adjoining the fluid path F must be comparatively readily biocompatible. In the embodiment shown, the entire fluid path F, or all components and/or portions adjoining the fluid path F, is/are biocompatible. This biocompatibility is guaranteed in this case by corresponding choice of material for the piston, wall and check valves.

To guarantee that the piston back 20 is pressurized with inlet-side fluid pressure p even in the upper end position of the piston 2, as shown in FIG. 1, the piston 2 has spacer portions 29. The spacer portions 29 lie on the partition wall 24, or more precisely on its underside, in the upper end position. The spacer portions 29 mean that the piston back 20 does not lie directly on the partition wall 24, and the portion K to this extent remains open for pressure transmission to the piston back 20.

In the embodiment shown, the downward movement of the piston 2 causes body fluid to be expelled from the pump volume V through the outlet A, and at the same time causes body fluid to be drawn into the inlet E as far as the portion K. During the upward movement of the piston 2, the body fluid initially conveyed into the portion K is drawn further into the pump volume V.

The pressure compensation explained above is advantageous for example if the ambient pressure (atmospheric pressure) of the patient changes. Such a change in ambient pressure naturally has an effect on the pressure of the body fluid. Its pressure corresponds to ambient pressure. In some situations, substantial pressure changes may occur, for example during air travel. In an aircraft cabin, the pressure may be up to 400 mbar lower than on the ground. The lower pressure accordingly is present on the inlet and outlet side of the pump device and hence also in the pump volume V. The pressure of the inner chamber 23 however remains constant, since this is encapsulated against the exterior. Accordingly, a pressure difference is created.

The action of the pressure difference on the entire movable piston 2 would result in a force which would make return of the piston 2 to the starting position more difficult. In order to counter this, higher return forces may be provided via an adaptation of the spring hardness of the piston 2 and/or the spring element 12. Such higher return forces are, however, disadvantageous under normal ambient pressure conditions. Then correspondingly higher pump forces would have to be applied, which would lead to a greater energy consumption.

Forces from the prevailing pressure differences are in this case reduced, in particular in that the partition wall 24 has been provided, dividing the interior of the housing 21 into a pressure-constant region 23 and a pressure-adapted region 22. The force resulting from said pressure difference now acts primarily on the surface of the sealing disc 26 and not on the entire piston 2. The required pump forces are therefore much less dependent on ambient pressure, since this pressure is present on both sides of the piston 2.

1. An implantable pump device for pumping a body fluid, the implantable pump device comprising:
a piston which is reciprocatingly movable along a stroke axis with a piston head,
a wall lying opposite the piston head along the stroke axis, and having
a pump volume which is enclosed between the piston head and the wall and forms a portion of a fluid path extending between an inlet and an outlet,
wherein a stroke movement of the piston causes a change in pump volume and accordingly a pump delivery of body fluid between the inlet and the outlet,
wherein the piston has a disc portion that is form-stable, an underside of the disc portion forming the piston head, and an elastic ring collar portion, wherein the elastic ring collar portion protrudes radially from the disc portion, at an inner periphery of the elastic ring collar portion is fixedly and fluid-tightly connected to an outer periphery of the disc portion, and at an outer periphery of the elastic ring collar portion is fixedly and fluid-tightly connected to the wall,
whereby the disc portion rests via the elastic ring collar portion on the wall so as to be reciprocatingly movable relative thereto and is fluid-tightly connected thereto.

2. The implantable pump device according to claim 1, wherein the disc portion and the elastic ring collar portion are formed integrally and cohesively with one another.

3. The implantable pump device according to claim 1, wherein the disc portion and the elastic ring collar portion are each made of metal.

4. The implantable pump device according to claim 3, wherein the metal is titanium.

5. The implantable pump device according to claim 1, wherein the outer periphery of the elastic ring collar portion is joined fixedly and fluid-tightly to the wall by a substance-bonded joint connection.

6. The implantable pump device according to claim 5, wherein the substance-bonded joint connection is a weld connection.

7. The implantable pump device according to claim 6, wherein the weld connection is produced without a welding additive.

8. The implantable pump device according to claim 1, wherein a thickness and/or a shaping of the elastic ring collar portion causes a spring force opposite the stroke movement, wherein the spring force causes or at least supports a complete return of the piston.

9. The implantable pump device according to claim 1, wherein the elastic ring collar portion has a flat shaping.

10. The implantable pump device according to claim 1, wherein the elastic ring collar portion has an undulating shaping.

11. The implantable pump device according to claim 1, wherein the wall has a further elastic ring collar portion, the outer periphery of the further elastic ring collar portion being fixedly and fluid-tightly connected to the outer periphery of the elastic ring collar portion of the piston.

12. The implantable pump device according to claim 11, wherein the elastic ring collar portion of the piston and the further elastic ring collar portion of the wall are arranged and/or configured mirror-symmetrically relative to a radial center longitudinal plane of the pump volume.

13. The implantable pump device according to claim 1, wherein the fluid path has a first check valve arranged between the inlet and the pump volume, and a second check valve arranged between the pump volume and the outlet, wherein the second check valve opens and closes in a direction opposite of the first check valve.

14. The implantable pump device according to claim 13, wherein the first check valve and/or the second check valve are integrated in the wall.

15. The implantable pump device according to claim 1, wherein a portion of the fluid path extending between the inlet and the pump volume adjoins a piston back of the piston which lies opposite the piston head along the stroke axis, whereby the piston back is pressurized with an inlet-side pressure of the body fluid.

16. The implantable pump device according to claim 15, further comprising a housing with a first inner chamber containing the piston, the wall and the fluid path, and with a second inner chamber which is sealed fluid-tightly against an environment and the first inner chamber and is configured to receive further components of the implantable pump device.

17. An implantable device comprising the implantable pump device according to claim 1.
